(19) United States
(12) Patent Application Publication
(10) Pub. No.: US 2011/0287074 A1
(43) Pub. Date: Nov. 24, 2011

(54) Antimicrobial Compositions, Methods of Manufacture Thereof and Articles Comprising the Same

(75) Inventors: Liwen Jin, Gainesville, FL (US); Ronald Howard Baney, Gainesville, FL (US)

(73) Assignee: UNIVERSITY OF FLORIDA RESEARCH FOUNDATION, INC., Gainesville, FL (US)

(21) Appl. No.: 13/114,250

(22) Filed: May 24, 2011

Related U.S. Application Data
(60) Provisional application No. 61/347,662, filed on May 24, 2010.

(51) Int. Cl. A61K 8/02; A61K 8/22; A61K 8/58; A61K 31/695; A61K 31/7048; A61K 36/31; A61K 36/28; A61K 38/02; A61Q 17/04; A61K 36/06; A61Q 19/02; A01N 55/10; A61L 9/00; A61Q 15/00; C07F 7/18; A01P 1/00; A61Q 19/08; A61P 17/00; A61P 17/10; A61P 39/06; A61P 31/00; A61P 31/10; A61Q 11/00; B05D 3/00; A61K 9/70; A61K 8/69

(52) U.S. Cl. 424/401; 424/52; 424/53; 424/49; 514/63; 514/27; 424/755; 424/764; 514/1.1; 424/59; 424/60; 424/195.16; 424/62; 424/76.2; 424/65; 556/463; 556/454; 556/453; 556/459; 427/2.29; 424/443

(57) ABSTRACT

Disclosed herein are a variety of applications for an antimicrobial agent. The antimicrobial agent comprises a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6.

### CROSS-REFERENCE TO RELATED APPLICATIONS

This application claims priority to U.S. Provisional Application No. 61/347,662 filed on May 24, 2010, the entire contents of which are hereby incorporated by reference.

### BACKGROUND

This disclosure relates to antimicrobial compositions, methods of manufacture thereof and articles comprising the same.

Antimicrobial personal care compositions are used to cleanse the skin and to destroy bacteria and other microorganisms present on the skin, especially the hands, arms, and face of the user. Antimicrobial compositions are used, for example, in the health care industry, food service industry, meat processing industry, and in the private sector by individual consumers. The widespread use of antimicrobial compositions indicates the importance consumers place on controlling bacteria and other microorganism populations on skin. It is desirable, however, that antimicrobial compositions provide a substantial and broad spectrum reduction in microorganism populations quickly and without problems associated with toxicity and skin irritation.

Most commercial antimicrobial compositions, however, generally offer a low to moderate antimicrobial activity. Antimicrobial activity is assessed against a broad spectrum of microorganisms, including both Gram positive and Gram negative microorganisms. The log reduction or alternatively the percent reduction, in bacterial populations provided by the antimicrobial composition correlates to antimicrobial activity. A log reduction of 3-5 is most desirable, a 1-3 reduction is desirable, whereas a log reduction of less than 1 is least desirable, for a particular contact time, generally ranging from 15 seconds to 5 minutes. Thus, a highly desirable antimicrobial composition exhibits a 3-5 log reduction against a broad spectrum of microorganisms in a short contact time. Prior disclosures illustrate attempts to provide such antimicrobial compositions, which, to date, do not provide the rapid, broad range control of microorganisms desired by consumers.

It should be noted that high log reductions have been achieved at pH values of 4 and 9, but such log reductions are attributed at least in part to these relatively extreme pH values. Compositions having such pH values can irritate the skin or damage other surfaces, and, therefore, are generally avoided. It has been difficult to achieve a high log reduction using an antimicrobial composition having a neutral pH of about 5.5 to about 7.5, and especially about 6 to about 7.3.

An efficacious antimicrobial composition having excellent esthetic properties has been difficult to achieve because of the chemical properties of the antimicrobial agents and the surfactants, and the effects of a surfactant on an antimicrobial agent. For example, nonionic and amphoteric surfactants do not provide a high foam level desired by consumers, and it is difficult to provide a phase stable, viscous composition. Anionic surfactants provide such properties, but cannot be formulated with quaternary ammonium antimicrobial agents.

Accordingly, a need exists for an antimicrobial composition that is highly efficacious against a broad spectrum of Gram positive and Gram negative bacteria in a short time period, and wherein the composition is viscous, phase stable, and provides consumer-acceptable esthetic properties, even in the absence of anionic surfactants. The present invention is directed to such antimicrobial compositions.

### SUMMARY

Disclosed herein is an oral care composition, comprising about 10% to about 99% of at least one ingredient selected from the group consisting of a polishing agent (abrasive agent), sudsing agents (surfactants), a binder, a humectant, a medicinal agent, peroxide sources, alkali metal bicarbonate salts, thickening materials, water, titanium dioxide, flavor agents, sweetening agents, xylitol, coloring agents, water and mixtures thereof; and an effective amount of an antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6.

Disclosed herein too is a personal care composition, comprising an effective amount of an antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6; a safe and effective amount of a second active agent, wherein said second active agent is selected from the group consisting of erythritol, p-cymen-7-ol, benzyl phenylacetate, 4-(4-methoxyphenyl)butan-2-one, ethoxyquin, tannic acid, gallic acid, octadecenedioic acid, p-cymen-5-ol, hesperedin, mustard seed extract, glycyrrhizic acid, glycyrrhetinic acid, carnosine, butylated hydroxytoluene, butylated hydroxyanisole, tetrahydrocurcumin, cetyl pyridinium chloride, ergothioneine, vanillin or its derivatives, diethylhexyl syrinylidene malonate, melanostatine, sterol esters, creatine, creatinine, feverfew extract, licochalcone A, sugar amine, vitamin B3 compounds, retinoids, peptides, phytosterol, dialkanoyl hydroxyproline, hexamidine compounds, salicylic acid, n-acyl amino acid compounds, sunscreen actives, water soluble vitamins, oil soluble vitamins, yeast cell derivative, and combinations thereof; an additional component, wherein the additional component is selected from the group consisting of desquamatory actives, anti-acne actives, wrinkle repair actives, anti-oxidants, radical scavengers, chelators, flavonoids, anti-inflammatory agents, anti-cellulite agents, tanning actives, skin lightening agents, antimicrobial actives, antifungal actives, conditioning agents, thickening agents, particulate material, topical anesthetics, and combinations thereof; and a dermatologically acceptable carrier.

Disclosed herein too is an adhesive latex formulation comprising a polymer; a thickening agent; and an effective amount of an antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6.

Disclosed herein too is a method comprising blending together a polymer; a thickening agent; and an effective amount of an antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6 to form a adhesive latex formulation composition; the adhesive latex formulation having a viscosity of at least 4,000 centipoise at 25° C. and at 1 atmosphere.

Disclosed herein too is a household treating agent comprising about 0.01 wt % to 20 wt % of an antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6.

Disclosed herein too is a deodorant composition comprising a deodorant; an antimicrobial agent; a polymer; and an aqueous or oily liquid medium; wherein the antimicrobial agent comprises a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6.

Disclosed herein too is a dental floss article comprising a filament; an antimicrobial agent; the antimicrobial agent being disposed upon the filament; the antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6.

Disclosed herein too is a dental floss composition comprising water; a surfactant; and an antimicrobial agent; the antimicrobial agent being disposed upon the filament; the antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6.

Disclosed herein too is a method comprising blending together water; a surfactant; and an antimicrobial agent to form a dental composition; the antimicrobial agent being disposed upon the filament; the antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6; applying the dental composition to the filament; wherein applying the dental composition to the filament is accomplished in a draw-dipping equipment; and drying the dental composition.

Disclosed herein too is a medical graft comprising a fabric material in a tubular shape; and an antimicrobial agent the antimicrobial agent being disposed upon the filament; the antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6.

### DETAILED DESCRIPTION

As used herein, the term “and/or” includes any and all combinations of one or more of the associated listed items.

The terminology used herein is for the purpose of describing particular embodiments only and is not intended to be limiting. As used herein, the singular forms “a,” “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The transition phrase “comprising” encompasses the transition phrases “consisting of” and “consisting essentially of”.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

All molecular weights expressed herein are number average molecular weights in grams per mole unless otherwise stated.

Disclosed herein is an antimicrobial composition that contains an antimicrobial agent. The antimicrobial composition may also include a surfactant, a thickening agent, pH adjusters, dyes, fragrances, skin conditioners and emollients, buffering agents, foam stabilizers, antioxidants, preservatives, foam enhancers, hydrotropes, water softening agents, chelating agents, opacifiers, and similar classes of optional ingredients.

The antimicrobial composition may be advantageously used as a skin cleanser, a body splash, a surgical scrub, a wound care agent, a hand sanitizer gel, a disinfectant, a mouth wash, a pet shampoo, a hard surface sanitizer, and the like. The antimicrobial composition may also be applied to a substrate (e.g., a filament, a garment, a cast, and the like) to form different articles. These articles will be listed below.

As noted above, the antimicrobial composition contains an antimicrobial agent. In one embodiment, the antimicrobial agent comprises a silanol-containing molecule. The silanol containing molecules can be selected from the group consisting of silanols having the structural formula (R₁R₂R₃SiOH), siloxanediols having the structural formula HO(R₁R₂SiO)ₙH and siloxanols having the structural formula HO(R₁R₂SiO)ₙSiR₁R₂R₃, where R₁, R₂ and R₃ are alkyl groups having 1 to about 6 carbon atoms, fluoroalkyl groups having 1 to about 6 carbon atoms, vinyl groups and/or aryl groups and n is less than 10.

In one exemplary embodiment, the silanol-containing molecule can be triethylsilanol, diphenylmethylsilanol, t-butyldimethylsilanol, n-butyldimethylsilanol, n-propyldimethylsilanol, ethyldimethylsilanol, vinylphenylmethylsilanol, phenyldimethylsilanol, 3,3,3 trifluoropropyldimethylsilanol, benzyldimethylsilanol and phenethyldimethylsilanol, or the like, or a combination comprising at least one of the foregoing molecules.

In another exemplary embodiment, the silanol containing molecule can be a fragrant silanol. In this embodiment, the fragrant silanol is selected from Ar(CH₂)ₐR₁R₂SiOH, where Ar is any phenyl or substituted phenyl group such that the substitution has a molecular weight of less than or equal to about 60, a is less than or equal to about 5, and R₁ and R₂ can be the same or different and are alkyl groups having less than or equal to about 4 carbon atoms, fluoro alkyl groups having less than or equal to about 4 carbon atoms or aryl groups. The fragrant silanol can be phenethyldimethylsilanol or benzyldimethylsilanol.

In one embodiment, the antimicrobial agent can be released in a controllable manner from hydrolysis of the silylated polymer. The silylation agent can be selected from commercial silylation agents such as Me₃SiNHSiMe₃, Me₃SiCl, Et₃SiNHSiEt₃, PhCH₂CH₂Me₂SiNHSiMe₂CH₂CH₂Ph, other similar precursors to the silanols described above, or a combination comprising at least one of the foregoing silylation agents. The silylated polymer is the reaction product from the hydroxyl containing polymer and the silylation agent; wherein the silylated polymer can be hydrolyzed to release silanols in a physiological environment.

The antimicrobial agent may be present in the antimicrobial composition in an amount of about 0.05 to about 20 weight percent (wt %), specifically about 1 to about 10 wt %, and more specifically about 2 to about 5 wt %, based on the total weight of the antimicrobial composition.

The antimicrobial composition comprises a surfactant composition. The surfactant composition may contain a silicone based surfactant and an optional cosurfactant. The silicone surfactants are those that can compatibilize the silicones with water or with an organic solvent. The surfactant does not impede the antimicrobial action of the antimicrobial agent.

In addition to an antimicrobial agent, the antimicrobial composition comprises a silicone containing surfactant. A preferred class of silicone containing surfactants are the polyalkylene oxide polysiloxanes having a dimethyl polysiloxane hydrophobic moiety and one or more hydrophilic polyalkylene side chains, and having the general formula (1):

R¹—(CH₃)₂SiO—[(CH₃)₂SiO]ₐ—[(CH₃)(R¹)SiO]_{b}—Si(CH₃)₂—R¹ (1)

wherein (a+b) is about 1 to about 50, specifically about 3 to about 30, more specifically about 10 to about 25, and each R¹ is the same or different and is selected from the group consisting of methyl and a poly(ethyleneoxide/propyleneoxide) copolymer group having the general formula (2):

—(CH₂)ₙO(C₂H₄O)_{c}(C₃H₆O)_{d}R² (2)

with at least one R¹ being a poly(ethyleneoxide/propyleneoxide) copolymer group, and wherein n is 3 or 4, specifically 3; total c (for all polyalkyleneoxy side groups) has a value of 1 to about 100, specifically from about 6 to about 100; total d is from 0 to about 14, specifically from 0 to about 3; and more specifically d is 0; total c+d has a value of from about 5 to about 150, specifically from about 9 to about 100 and each R² is the same or different and is selected from the group consisting of hydrogen, an alkyl having 1 to 4 carbon atoms, and an acetyl group, specifically hydrogen and methyl group. In one embodiment, each polyalkylene oxide polysiloxane has at least one R¹ group being a poly(ethyleneoxide/propyleneoxide) copolymer group.

Nonlimiting examples of these types of surfactants are the Silwet® surfactants which are available OSi Specialties, Inc., Danbury, Conn. Representative Silwet surfactants are as follows.

| | Average molecular | | |
|---|---|---|---|
| | weight | | |
| Name | (grams/mole) | Average a + b | Average total c |
| L-7608 | 600 | 1 | 9 |
| L-7607 | 1,000 | 2 | 17 |
| L-77 | 600 | 1 | 9 |
| L-7605 | 6,000 | 20 | 99 |
| L-7604 | 4,000 | 21 | 53 |
| L-7600 | 4,000 | 11 | 68 |
| L-7657 | 5,000 | 20 | 76 |
| L-7602 | 3,000 | 20 | 29 |

The molecular weight of the polyalkyleneoxy group (R¹) is less than or equal to about 10,000. Specifically, the molecular weight of the polyalkyleneoxy group is less than or equal to about 8,000 grams per mole, and most specifically is about 300 to about 5,000 grams per mole. Thus, the values of c and d can be those numbers, which provide molecular weights within these ranges. However, the number of ethyleneoxy units (—C₂H₄O) in the polyether chain (R¹) must be sufficient to render the polyalkylene oxide polysiloxane water dispersible or water soluble. If propyleneoxy groups are present in the polyalkylenoxy chain, they can be distributed randomly in the chain or exist as blocks. Preferred Silwet surfactants are L-7600, L-7602, L-7604, L-7605, L-7622, L-7657, and mixtures thereof. Besides surface activity, polyalkylene oxide polysiloxane surfactants can also provide other benefits, such as lubricity and skin softness.

The silicone containing surfactant is present in an amount of about 1 wt % to about 15 wt %, specifically about 1.5 wt % to about 10 wt %, and more specifically about 2 wt % to about 8 wt % of the antimicrobial composition.

As discussed above, cosurfactants may be used in the antimicrobial composition.

The cosurfactant can be (a) a nonionic surfactant, such as a polyoxyethylene alcohol condensate, an alkylpolyglucoside (APG) surfactant, and the like, (b) a cationic surfactant, such as an amine salt, a quaternary ammonium surfactant, an amidopropyl betaine monoethanolamide, and the like, or (c) a mixture thereof. Examples of cosurfactants include, but are not limited to, lauryl polyglucose, decyl polyglucose (e.g., PLANTAREN 2000N from Cognis Care Chemicals, Ambler, Pa.), cocamidopropylbetaine MEA chloride (MONTALAINE C40® from Seppic, Paris, France), and sunflower seed amidopropylethyldimonium ethosulfate (MACKERNIUM DY83®, McIntyre Chemical Co., University Park, Ill.). If the cosurfactant is a quaternary ammonium surfactant, such a surfactant does not exhibit the typical antimicrobial properties of a quaternary ammonium antimicrobial agent.

A nonionic cosurfactant has a hydrophobic base, such as a long chain alkyl group or an alkylated aryl group, and a hydrophilic chain comprising a ethoxy and/or propoxy moieties. The hydrophilic chain specifically contains ethoxy moieties. As defined herein, a “nonionic cosurfactant” has a hydrophobic base having an alkyl group containing six to eighteen carbon atoms, and an average of one to about twenty ethoxy and/or propoxy moieites. Examples of classes of nonionic cosurfactants include ethoxylated alkylphenols, ethoxylated and propoxylated fatty alcohols, alkylpolyglucosides, polyethylene glycol ethers of sorbitol, ethylene oxide-propylene oxide block copolymers, ethoxylated esters of fatty (C₈-C₁₄) acids, condensation products of ethylene oxide with long chain amines or amides, and mixtures thereof. Suitable block polyoxyethylene-polyoxypropylene polymeric cosurfactants include those based on ethylene glycol, propylene glycol, glycerol, trimethylolpropane and ethylenediamine may be used in the antimicrobial composition. Polymeric compounds made from a sequential ethoxylation and propoxylation of initial compounds with a single reactive hydrogen atom, such as C₁₂₋₁₈ aliphatic alcohols, are not generally compatible with certain odor controlling agents (e.g., cyclodextrin). Certain of the block polymer surfactant compounds designated Pluronic® and Tetronic® by the BASF-Wyandotte Corp., Wyandotte, Mich., are readily available. Nonlimiting examples of suitable surfactants of this type include pluronic surfactants with the general formula H(EO)ₙ(PO)ₘ(EO)ₙH, wherein EO is an ethylene oxide group, PO is a propylene oxide group, and n and m are numbers that indicate the average number of the groups in the surfactants.

Examples of nonionic cosurfactants include, but are not limited to, C₁₁-C₁₅ pareth-20, ceteth-12, dodoxynol-12, laureth-15, polysorbate 20, an ethoxylated nonylphenol, ethoxylated octylphenol, ethoxylated dodecylphenol, or ethoxylated fatty (C₆-C₁₈) alcohol, including 4 to 20 ethylene oxide moieties, glycereth-12, trideceth-9, trideceth-10, trideceth-11, trideceth-12, trideceth-15, sorbeth-20, dodoxynol-9, dodoxynol-12, chlorodeceth-14, chloeth-10, dihydrocholeth-15, isoceteth-10, isoceteth-20, isolaureth-10, isosteareth-10, isosteareth-12, isosteareth-20, laneth-10, laneth-15, laneth-16, laneth-20, oleth-9, oleth-10, oleth-12, oleth-15, oleth-16, oleth-20, steareth-10, steareth-11, steareth-13, steareth-15, steareth-16, steareth-20, talloweth-6, laureth-9, laureth-10, laureth-11, laureth-12, laureth-13, laureth-14, laureth-15, laureth-20, and mixtures thereof.

Numerous other nonionic surfactants are disclosed in McCutcheon's Detergents and Emulsifiers, 1993 Annuals, published by McCutcheon Division, MC Publishing Co., Glen Rock, N.J., pp. 1-246 and 266-273; in the CTFA International Cosmetic Ingredient Dictionary, Fourth Ed., Cosmetic, Toiletry and Fragrance Association, Washington, D.C. (1991) (hereinafter the CTFA Dictionary) at pages 1-651; and in the CTFA Cosmetic Ingredient Handbook, First Ed., Cosmetic, Toiletry and Fragrance Association, Washington, D.C. (1988) (hereafter the CTFA Handbook), at pages 86-94, each incorporated herein by reference.

Cationic cosurfactants include a quaternary surfactant having a structural formula (3)

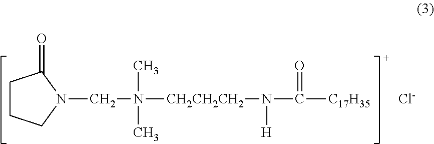

a quaternized phosphate ester, such as PHOSPHOLIPID SV, available from Mona Industries, Paterson, N.J., e.g., stearamidopropyl phosphatidyl PG-dimonium chloride, linoleamidopropyl phosphatidyl PG-dimonium chloride, coco phosphatidyl PG-dimonium chloride, cocamidopropyl phosphatidyl PG-dimonium chloride, borageamidopropyl phosphatidyl PG-dimonium chloride, and cocohydroxyethyl phosphatidyl PG-imidazolinium chloride; and other quaternized phosphate esters disclosed in U.S. Pat. No. 4,209,449 to Mayhew et al. Additional quaternary ammonium surfactants can be found in the CTFA Handbook at pages 40-42, incorporated herein by reference.

In addition to the antimicrobial agent and the surfactant, the antimicrobial composition contains about 1% to about 10%, and specifically about 1.5% to about 8%, by weight, of a cosurfactant. In one embodiment, the composition contains about 2% to about 6%, by weight of the composition, of a cosurfactant.

The antimicrobial composition optionally can contain a thickener. The polymeric thickener can be (a) a cellulose thickener, (b) a hydrophobically modified polyethylene glycol (PEG) or hydrophobically modified polypropylene glycol (PPG), or (c) a hydrophobic alkoxylated alcohol. A cellulose thickener can be a nonionic cellulose or a cationic cellulose. Useful cellulose thickeners include hydroxyethylcellulose, hydroxybutyl methylcellulose, hydroxypropyl methylcellulose, hydroxypropylcellulose, polyquaternium 10, polyquaternium 4, hydroxypropyl methylcellulose, and hydroxyethyl ethylcellulose.

A hydrophobic PEG, PPG, or alkoxylated alcohol contains a hydrophobic hydrocarbon moiety, either alkyl or alkylated aryl. The alkyl group of the hydrophobic moiety contains fourteen carbon atoms, or more (e.g., up to thirty carbon atoms). Such thickeners further contain an average of at least twenty ethoxy and/or propoxy moieties.

Examples of a hydrophobically modified PEG or hydrophobically modified PPG include PEG-20 through PEG-200, or PPG-20 through PPG-34, either having a hydrophobic moiety attached thereto or copolymerized with a hydrophobic monomer. The hydrophobic monomer can be a C₁₄-C₁₈ glycol, for example. The hydrophobic moiety can be an alkylated phenol residue, such as a nonylphenol residue, a fatty acid residue, a fatty amide residue, a fatty amine residue, and similar residues having a long-chain alkyl (e.g., C₁₄-C₂₂) and/or an aryl component. Examples of an alkoxylated alcohol include a C₁₄-C₂₀ alcohol alkoxylate with 20 to 100 moles of ethylene oxide and/or propylene oxide, and having a hydrophobic moiety present in the molecule.

Specific examples of hydrophobically modified polymeric thickeners include PEG-120 methyl glucose dioleate, PPG-14 palmeth-60 alkyl, ceteareth-60 myristyl glycol, methoxy PEG-22/dodecyl glycol copolymer, methyl gluceth-20, PEG-20 castor oil, PEG-25 castor oil, PEG-30 castor oil, PEG-36 castor oil, PEG-40 castor oil, PEG-50 castor oil, PEG-60 castor oil, PEG-100 castor oil, PEG-45/dodecyl glycol copolymer, PEG-20 hydrogenated castor oil, PEG-25 hydrogenated castor oil, PEG-30 hydrogenated castor oil, PEG-40 hydrogenated castor oil, PEG-50 hydrogenated castor oil, PEG-60 hydrogenated castor oil, PEG-80 hydrogenated castor oil, PEG-100 hydrogenated castor oil, PPG-20 lanolin alcohol PPG-30 lanolin alcohol ether, PPG-25-laureth-25, PPG-20 oleyl ether, PPG-23 oleyl ether, PPG-30 oleyl ether, PPG-37 oleyl ether, PPG-50 oleyl ether, PPG-20 methyl glucose ether, PPG-20 methyl glucose ether acetate, PEG-20 lanolin, PEG-24 lanolin, PEG-27 lanolin, PEG-30 lanolin, PEG-40 lanolin, PEG-50 lanolin, PEG-60 lanolin, PEG-75 lanolin, PEG-85 lanolin, PEG-10 lanolin, PEG-75 lanolin oil, PEG-75 lanolin wax, PEG-20 methyl glucose sesquistereate, PEG-20-PPG-10 glyceryl stearate, PEG-25 propylene glycol stearate, PEG-75 propylene glycol stearate, PEG-120 propylene glycol stearate, PEG-25 soya sterol, PEG-40 soya sterol, talloweth-60 myristyl glycol, or the like, or a combination comprising at least one of the foregoing polymeric thickeners.

A polymeric thickener is present in a sufficient amount to provide a composition having a viscosity of about 100 to about 10,000 centipoise, specifically about 300 to about 6000 centipoise, and more specifically about 500 to about 5000 centipoise. In an exemplary embodiment, an antimicrobial composition has a viscosity of about 2000 to about 5000 centipoise. In the absence of a polymeric thickener, the antimicrobial composition has a viscosity of about 0.1 to about 100 centipoise.

The polymeric thickener is present in an amount of 0 wt % to about 5 wt %, and specifically 0.25 wt % to about 4 wt %, of the composition. In one embodiment, the thickener can be present in an amount of about 0.5 wt % to about 3 wt %, of the antimicrobial composition.

Examples of basic pH adjusters are ammonia; mono-, di-, and tri-alkyl amines; mono-, di-, and tri-alkanolamines; alkali metal and alkaline earth metal hydroxides; and mixtures thereof. However, the identity of the basic pH adjuster is not limited, and any basic pH adjuster alone or in combination, can be used. Examples of basic pH adjusters are ammonia; sodium, potassium, and lithium hydroxide; monoethanolamine; triethylamine; isopropanolamine; diethanolamine; and triethanolamine.

Examples of acidic pH adjusters are the mineral acids and polycarboxylic acids. Non limiting examples of mineral acids are hydrochloric acid, nitric acid, phosphoric acid, and sulfuric acid. Non limiting examples of polycarboxylic acids are citric acid, glycolic acid, and lactic acid. The identity of the acidic pH adjuster is not limited and any acidic pH adjuster known in the art, alone or in combination, can be used.

With respect to pH adjusters, the antimicrobial composition has a pH of about 5.5 to about 7.5 to provide a high, broad-spectrum antimicrobial efficacy. An optional pH adjuster can be used in a sufficient amount to provide a pH of about 5.5 to about 7.5, or a preferred pH of about 6 to about 7.3.

An antimicrobial composition of the present invention also can contain optional ingredients known to persons skilled in the art. For example, the composition can contain a hydric solvent and/or a hydrotrope. The present compositions also can contain other optional ingredients, such as skin conditioners, dyes, and fragrances, that are present in a sufficient amount to perform their intended function and do not adversely affect the antimicrobial efficacy or consumer acceptance of the composition. Such optional ingredients typically are present, individually, from 0% to about 5%, by weight, of the composition, and, collectively, from 0% to about 20%, by weight, of the composition.

The antimicrobial composition may also contain whole classes of optional ingredients. These include, but are not limited to, dyes, fragrances, pH adjusters, skin conditioners and emollients, buffering agents, foam stabilizers, antioxidants, preservatives, foam enhancers, hydrotropes, water softening agents, chelating agents, opacifiers, and similar classes of optional ingredients.

Specific optional ingredients include alkanolamides as foam boosters and stabilizers; inorganic phosphates, sulfates, and carbonates as buffering agents; mono-, di-, and triglycerides (e.g., glycerol monolaurate) as opacifiers, viscosity modifiers, or skin conditioners; EDTA and phosphates as chelating agents.

An alkanolamide used to provide foam enhancement and foam stability can be, but is not limited to, cocamide MEA, cocamide DEA, soyamide DEA, lauramide DEA, oleamide MIPA, stearamide MEA, myristamide MEA, lauramide MEA, capramide DEA, ricinoleamide DEA, myristamide DEA, stearamide DEA, oleylamide DEA, tallowamide DEA, lauramide MIPA, tallowamide MEA, isostearamide DEA, isostearamide MEA, or the like, or a combination comprising at least one of the foregoing alkanolamides.

Optional skin conditioners and emollients include, but are not limited to, an ester having at least 10 carbon atoms, and specifically 10 to about 32 carbon atoms. Suitable esters include those comprising an aliphatic alcohol having about 8 to about 20 carbon atoms and an aliphatic or aromatic carboxylic acid including 2 to about 12 carbon atoms, or conversely, an aliphatic alcohol having 2 to about 12 carbon atoms with an aliphatic or aromatic carboxylic acid including 8 to about 20 carbon atoms. The ester is either straight chained or branched. Specifically, the ester has a molecular weight of less than about 500 and provides emollient properties.

Suitable esters, therefore, include, for example, but are not limited to (a) aliphatic monohydric alcohol esters, including, but not limited to, myristyl propionate, isopropyl isostearate, isopropyl myristate, isopropyl palmitate, cetyl acetate, cetyl propionate, cetyl stearate, isodecyl neopentanoate, cetyl octanoate, and isocetyl stearate; (b) aliphatic di- and triesters of polycarboxylic acids, including, but not limited to, diisopropyl adipate, diisostearyl fumarate, dioctyl adipate, and triisostearyl citrate; (c) aliphatic polyhydric alcohol esters, including, but not limited to, propylene glycol dipelargonate; (d) aliphatic esters of aromatic acids, including, but not limited to, C₁₂-C₁₅ alcohol esters of benzoic acid, octyl salicylate, sucrose benzoate, and dioctyl phthalate. Numerous other esters are listed in the CTFA Handbook at pages 24 through 26, incorporated herein by reference.

The antimicrobial composition also optionally can include an oil. Examples of oils that can be included in the composition include, but are not limited to, apricot kernel oil, avocado oil, C₃₀-C₄₆ piscine oil, castor oil, chaulmoogra oil, cherry pit oil, coconut oil, corn oil, cottonseed oil, egg oil, ethiodized oil, grape seed oil, hazel nut oil, hybrid safflower oil, lanolin oil, linseed oil, menhaden oil, mink oil, moring a oil, neatsfoot oil, olive husk oil, olive oil, palm kernel oil, palm oil, peach kernel oil, peanut oil, pengawar djambi oil, rapeseed oil, rice bran oil, safflower oil, sesame oil, soybean oil, sunflower seed oil, sweet almond oil, walnut oil, wheat germ oil, cod liver oil, hydrogenated castor oil, hydrogenated coconut oil, hydrogenated cottonseed oil, hydrogenated jojoba oil, hydrogenated menhaden oil, hydrogenated palm kernel oil, white petrolatum, hydrogenated palm oil, hydrogenated peanut oil, hydrogenated shark liver oil, hydrogenated soybean oil, hydrogenated vegetable oil, jojoba oil, shark liver oil, synthetic jojoba oil, tall oil, vegetable oil, bay oil, cottonseed oil, or the like, or a combination comprising at least one of the foregoing oils.

The antimicrobial composition can also contain 0 wt % to about 20 wt % of a hydric solvent, and 0% to about 20%, by weight, of a hydrotrope. As used herein, the term “hydric solvent” is defined as a water-soluble organic compound containing one to six, and typically one to three, hydroxyl groups. The term “hydric solvent” therefore encompasses water-soluble alcohols, diols, triols, and polyols. Specific examples of hydric solvents include, but are not limited to, methanol, ethanol, isopropyl alcohol, n-butanol, n-propyl alcohol, ethylene glycol, propylene glycol, glycerol, diethylene glycol, dipropylene glycol, tripropylene glycol, hexylene glycol, butylene glycol, 1,2,6-hexanetriol, sorbitol, PEG-4, or the like, or a combination comprising at least one of the foregoing hydric solvents.

A hydrotrope is a compound that has the ability to enhance the water solubility of other compounds. An example of an optional hydrotrope is a short-chain alkyl aryl sulfonate. Specific examples of hydrotropes include, but are not limited to, sodium cumene sulfonate, ammonium cumene sulfonate, ammonium xylene sulfonate, potassium toluene sulfonate, sodium toluene sulfonate, sodium xylene sulfonate, toluene sulfonic acid, and xylene sulfonic acid. Other useful hydrotropes include sodium polynaphthalene sulfonate, sodium polystyrene sulfonate, sodium methyl naphthalene sulfonate, disodium succinate, or the like, or a combination comprising at least one of the foregoing hydrotropes.

The antimicrobial composition also can contain a preservative in an amount of 0% to about 0.5 wt % by weight. Examples of preservatives include, but are not limited to, sorbic acid, potassium sorbate, the parabens (like benzylparaben), imidazolinylurea, methylchloroisothiazolinone, and the hydantoins, like DMDM hydantoin. Additional preservatives as disclosed in the CTFA Handbook at page 78, incorporated herein by reference.

A present antimicrobial composition further can contain an antioxidant and/or an ultraviolet light (UV) absorber, each independently in an amount of 0% to about 0.5% by weight. Examples of antioxidants and UV absorbers include, but are not limited to, BHA, BHT, sodium ascorbate, potassium sulfite, erythorbic acid, benzophenone-1 through benzophenone-12, and PABA. Additional antioxidants and UV absorbers can be found in the CTFA Handbook at pages 78 and 98, incorporated herein by reference.

The carrier of the antimicrobial composition comprises water. An antimicrobial composition does not rely upon a low pH or a high pH to provide a rapid reduction in bacterial populations. Antimicrobial compositions have a pH of about 5.5 to about 7.5, and specifically about 6 to about 7.3. Within this pH range, the present compositions effectively reduce Gram positive and Gram negative bacterial populations, and are consumer acceptable, i.e., have a consumer acceptable viscosity, are mild to the skin, are phase stable, and generate a high, stable foam. Such results are surprising for an antimicrobial composition that is free of an anionic surfactant.

The antimicrobial composition may contain a variety of different organic polymers. The organic polymers can be selected from a wide variety of thermoplastic polymers, blends of thermoplastic polymers, thermosetting polymers, or blends of thermoplastic polymers with thermosetting polymers. The organic polymer may also be a blend of polymers, copolymers, terpolymers, or combinations comprising at least one of the foregoing organic polymers. The organic polymer can also be an oligomer, a homopolymer, a copolymer, a block copolymer, an alternating block copolymer, a random polymer, a random copolymer, a random block copolymer, a graft copolymer, a star block copolymer, a dendrimer, or the like, or a combination comprising at last one of the foregoing organic polymers.

Examples of the organic polymers are polyacetals, polyolefins, polyacrylics, polycarbonates, polystyrenes, polyesters, polyamides, polyamideimides, polyarylates, polyarylsulfones, polyethersulfones, polyphenylene sulfides, polyvinyl chlorides, polysulfones, polyimides, polyetherimides, polytetrafluoroethylenes, polyetherketones, polyether etherketones, polyether ketone ketones, polybenzoxazoles, polyphthalides, polyacetals, polyanhydrides, polyvinyl ethers, polyvinyl thioethers, polyvinyl alcohols, polyvinyl ketones, polyvinyl halides, polyvinyl nitriles, polyvinyl esters, polysulfonates, polysulfides, polythioesters, polysulfones, polysulfonamides, polyureas, polyphosphazenes, polysilazanes, styrene acrylonitrile, acrylonitrile-butadiene-styrene (ABS), polyethylene terephthalate, polybutylene terephthalate, polyurethane, ethylene propylene diene rubber (EPR), polytetrafluoroethylene, fluorinated ethylene propylene, perfluoroalkoxyethylene, polychlorotrifluoroethylene, polyvinylidene fluoride, or the like, or a combination comprising at least one of the foregoing organic polymers.

Examples of thermosetting polymers suitable for use in the polymeric composition include epoxy polymers, unsaturated polyester polymers, polyimide polymers, bismaleimide polymers, bismaleimide triazine polymers, cyanate ester polymers, vinyl polymers, benzoxazine polymers, benzocyclobutene polymers, acrylics, alkyds, phenol-formaldehyde polymers, novolacs, resoles, melamine-formaldehyde polymers, urea-formaldehyde polymers, hydroxymethylfurans, isocyanates, diallyl phthalate, triallyl cyanurate, triallyl isocyanurate, unsaturated polyesterimides, or the like, or a combination comprising at least one of the foregoing thermosetting polymers.

Examples of blends of thermoplastic polymers include acrylonitrile-butadiene-styrene/nylon, polycarbonate/acrylonitrile-butadiene-styrene, acrylonitrile butadiene styrene/polyvinyl chloride, polyphenylene ether/polystyrene, polyphenylene ether/nylon, polysulfone/acrylonitrile-butadiene-styrene, polycarbonate/thermoplastic urethane, polycarbonate/polyethylene terephthalate, polycarbonate/polybutylene terephthalate, thermoplastic elastomer alloys, nylon/elastomers, polyester/elastomers, polyethylene terephthalate/polybutylene terephthalate, acetal/elastomer, styrene-maleicanhydride/acrylonitrile-butadiene-styrene, polyether etherketone/polyethersulfone, polyether etherketone/polyetherimide polyethylene/nylon, polyethylene/polyacetal, or the like.

A number of different applications of the antimicrobial agent are described below. These applications all involve the silanol containing molecule. Other ingredients in the respective applications may include some of the ingredients listed above or other ingredients not particularly listed above.

The applications involve fluids, slurries, pastes, dispersions, and the like that may be applied to the body if living beings. In one embodiment, the fluids, slurries, pastes, dispersions, and the like, may be applied to a substrate in the form of a coating. The substrate may include grafts, clothes, wrappings, casts, and the like. The graft, clothes, wrappings, casts, and the like, may be applied to the body of a living being, or applied to the surface of a device (e.g., furniture, hospital doors, handles, toilets seats, and the like) and removed when desired

### Oral Care Composition

In one embodiment the antimicrobial agent may be used in an oral care composition. The antimicrobial agent comprises the silanol containing molecule selected from the group consisting of silanols, siloxanediols and a combination comprising at least one of the foregoing silanol containing molecules. The oral care composition further comprises about 10% to about 99% of at least one ingredient selected from the group consisting of a polishing agent (abrasive agent), sudsing agents (surfactants), a binder, a humectant, a medicinal agent, peroxide sources, alkali metal bicarbonate salts, thickening materials, water, titanium dioxide, flavor agents, sweetening agents, xylitol, coloring agents, water and mixtures thereof.

In one embodiment, the oral care composition may include the antimicrobial agent being released from a silylated polymer. The antimicrobial agent can be released in a controllable manner from the hydrolysis of a silylated polymer. The antimicrobial agent can be in dispersed or bulk form in the silylated polymer. The polymer can be a hydroxyl-containing polymer, where the hydroxyl-containing polymer is a polysaccharide, a protein, a polyalkylene glycol, a hydroxyl-terminated polymer, an amine-terminated polymer, or a combination comprising at least one of the foregoing polymers.

In one embodiment, the antimicrobial agent can be released in a controllable manner by hydrolysis of the silylated polymer. The silylation agent can be selected from commercial silylation agents such as Me₃SiNHSiMe₃, Me₃SiCl, Et₃SiNHSiEt₃, PhCH₂CH₂Me₂SiNHSiMe₂CH₂CH₂Ph, other similar precursors to the silanols described above, or a combination comprising at least one of the foregoing silylation agents. The silylated polymer is the reaction product from the hydroxyl containing polymer and the silylation agent; wherein the silylated polymer can be hydrolyzed to release silanols in a physiological environment.

### Personal Care Composition

In another embodiment, the antimicrobial composition containing the antimicrobial agent may be used in a personal care composition. The antimicrobial agent comprises the silanol containing molecule selected from the group consisting of silanols, siloxanediols and a combination comprising at least one of the foregoing silanol containing molecules.

The personal care composition contains a safe and effective amount of a second active agent, wherein the second active agent is selected from the group consisting of erythritol, p-cymen-7-ol, benzyl phenylacetate, 4-(4-methoxyphenyl)butan-2-one, ethoxyquin, tannic acid, gallic acid, octadecenedioic acid, p-cymen-5-ol, hesperedin, mustard seed extract, glycyrrhizic acid, glycyrrhetinic acid, carnosine, butylated hydroxytoluene (BHT) and butylated hydroxyanisole (BHA), tetrahydrocurcumin, cetyl pyridinium chloride, ergothioneine, vanillin or its derivatives, diethylhexyl syrinylidene malonate, melanostatine, sterol esters, creatine, creatinine, feverfew extract, licochalcone A, sugar amine, vitamin B3 compounds, retinoids, peptides, phytosterol, dialkanoyl hydroxyproline, hexamidine compounds, salicylic acid, n-acyl amino acid compounds, sunscreen actives, water soluble vitamins, oil soluble vitamins, yeast cell derivative, and combinations thereof and optionally, an additional component selected from the group consisting of desquamatory actives, anti-acne actives, wrinkle repair actives, anti-oxidants, radical scavengers, chelators, flavonoids, anti-inflammatory agents, anti-cellulite agents, tanning actives, skin lightening agents, antimicrobial actives, antifungal actives, conditioning agents, thickening agents, particulate material, topical anesthetics, and combinations thereof; and optionally, a dermatologically acceptable carrier.

In one embodiment, the personal care compositions may include the antimicrobial agent being released from a silylated polymer. The antimicrobial agent can be released in a controllable manner from the hydrolysis of a silylated polymer. The antimicrobial agent can be in dispersed or bulk form in the silylated polymer. The polymer can be a hydroxyl-containing polymer, where the hydroxyl-containing polymer is a polysaccharide, a protein, a polyalkylene glycol, a hydroxyl-terminated polymer, an amine-terminated polymer, or a combination comprising at least one of the foregoing polymers.

The antimicrobial agent can be released in a controllable manner by hydrolysis of the silylated polymer. The silylation agent can be selected from commercial silylation agents such as Me₃SiNHSiMe₃, Me₃SiCl, Et₃SiNHSiEt₃, PhCH₂CH₂Me₂SiNHSiMe₂CH₂CH₂Ph, other similar precursors to the silanols described above, or a combination comprising at least one of the foregoing silylation agents. The silylated polymer is the reaction product from the hydroxyl containing polymer and the silylation agent; wherein the silylated polymer can be hydrolyzed to release silanols in a physiological environment.

### Deodorant and Antimicrobial Dispersion

In one embodiment, the antimicrobial agent may be used in a deodorant or in an antimicrobial dispersion. The deodorant may be used on the skin, in the armpits, in the groins, and other parts of the bodies of living beings.

In one embodiment, the deodorant composition comprises a deodorant; an antimicrobial agent; a polymer; and an aqueous or oily liquid medium. The antimicrobial agent comprises the silanol containing molecule selected from the group consisting of silanols, siloxanediols and a combination comprising at least one of the foregoing silanol containing molecules. The antimicrobial agent may be present in the deodorant or antimicrobial dispersion in an amount of about 0.01 to about 5 wt % of the total weight of the deodorant or the antimicrobial dispersion.

The polymer is used as a binder and suitable polymers may be found in the list above. The deodorant may further contain a dermatologically acceptable fragrance. Dermatologically acceptable carriers may also be used in the deodorant and in the antimicrobial dispersion.

In one embodiment, the deodorants may include the antimicrobial agent being released from a silylated polymer. The antimicrobial agent can be released in a controllable manner from the hydrolysis of a silylated polymer. The antimicrobial agent can be in dispersed or bulk form in the silylated polymer. The polymer can be a hydroxyl-containing polymer, where the hydroxyl-containing polymer is a polysaccharide, a protein, a polyalkylene glycol, a hydroxyl-terminated polymer, an amine-terminated polymer, or a combination comprising at least one of the foregoing polymers.

The antimicrobial agent can be released from the deodorant in a controllable manner by hydrolysis of the silylated polymer. The silylation agent can be selected from commercial silylation agents such as Me₃SiNHSiMe₃, Me₃SiCl, Et₃SiNHSiEt₃, PhCH₂CH₂Me₂SiNHSiMe₂CH₂CH₂Ph, other similar precursors to the silanols described above, or a combination comprising at least one of the foregoing silylation agents. The silylated polymer is the reaction product from the hydroxyl containing polymer and the silylation agent; wherein the silylated polymer can be hydrolyzed to release silanols in a physiological environment.

### Emulsions

The antimicrobial agent may also be used in an emulsion. The emulsion may be used in a variety of body care products such as shampoos, hair care products, skin care products, dentrifices, deodorizing preparations, antiperspirant preparations, and the like.

The emulsion may contain about 0.01 to about 20 wt % of the antimicrobial agent, based on the total weight of the emulsion. The antimicrobial agent comprises the silanol containing molecule selected from the group consisting of silanols, siloxanediols and a combination comprising at least one of the foregoing silanol containing molecules.

The emulsion may further contain about 0.1 to about 5 wt % of a chelating agent. The chelating agent may be selected from the group consisting of ethylenediamine, tetraacetic acid and salts thereof, lactic acid, acidic polyphosphates, or the like, or a combination comprising at least one of the foregoing chelating agent. The emulsion may also contain about 20 to 70 wt % of a surfactant, about 1 to about 10 wt % of sodium isostearoyl lactylate as an emulsifier, about 1 to about 10 wt % of a moisturizer selected from the group consisting of lactic acid and sodium lactate and about 25 to about 75 wt % water, based on the total weight of the emulsion. In one embodiment, when the emulsion is used as a shampoo, the antimicrobial agent is triethylsilanol comprising about 0.1 to about 15 wt % of the emulsion.

In one embodiment, the emulsions may include the antimicrobial agent being released from a silylated polymer. The antimicrobial agent can be released in a controllable manner from the hydrolysis of a silylated polymer. The antimicrobial agent can be in dispersed or bulk form in the silylated polymer. The polymer can be a hydroxyl-containing polymer, where the hydroxyl-containing polymer is a polysaccharide, a protein, a polyalkylene glycol, a hydroxyl-terminated polymer, an amine-terminated polymer, or a combination comprising at least one of the foregoing polymers.

The antimicrobial agent can be released from the deodorant in a controllable manner by hydrolysis of the silylated polymer. The silylation agent can be selected from commercial silylation agents such as Me₃SiNHSiMe₃, Me₃SiCl, Et₃SiNHSiEt₃, PhCH₂CH₂Me₂SiNHSiMe₂CH₂CH₂Ph, other similar precursors to the silanols described above, or a combination comprising at least one of the foregoing silylation agents. The silylated polymer is the reaction product from the hydroxyl containing polymer and the silylation agent; wherein the silylated polymer can be hydrolyzed to release silanols in a physiological environment.

### Household Treating Agents

The antimicrobial agent may be advantageously used in household treating compositions. The household compositions may be selected from the group consisting of but not limited to decorative preparations, laundry detergents and fabric softeners, non-detergent based fabric care products, liquid cleansing and scouring agents, glass detergents, neutral cleaners, all-purpose cleaners, acid household cleaners, bathroom cleaners, dishwashing agents, kitchen and oven cleaners, clear rinsing agents, dishwasher detergents, shoe polishes, polishing waxes, floor detergents and polishes, metal, glass, and ceramic cleaners, fabric care products, rug cleaners and carpet shampoos, agents for removing rust, furniture and multipurpose polishes, leather and vinyl dressing agents, solid and liquid air fresheners, and the like.

The antimicrobial agent may be present in an amount of about 0.01 to about 20 wt % based on the total formulation of any of the house treating agents. The antimicrobial agent comprises the silanol containing molecule selected from the group consisting of silanols, siloxanediols and a combination comprising at least one of the foregoing silanol containing molecules.

In one embodiment, the house treating agents may include the antimicrobial agent being released from a silylated polymer. The antimicrobial agent can be released in a controllable manner from the hydrolysis of a silylated polymer. The antimicrobial agent can be in dispersed or bulk form in the silylated polymer. The polymer can be a hydroxyl-containing polymer, where the hydroxyl-containing polymer is a polysaccharide, a protein, a polyalkylene glycol, a hydroxyl-terminated polymer, an amine-terminated polymer, or a combination comprising at least one of the foregoing polymers.

The antimicrobial agent can be released from the deodorant in a controllable manner by hydrolysis of the silylated polymer. The silylation agent can be selected from commercial silylation agents such as Me₃SiNHSiMe₃, Me₃SiCl, Et₃SiNHSiEt₃, PhCH₂CH₂Me₂SiNHSiMe₂CH₂CH₂Ph, other similar precursors to the silanols described above, or a combination comprising at least one of the foregoing silylation agents. The silylated polymer is the reaction product from the hydroxyl containing polymer and the silylation agent; wherein the silylated polymer can be hydrolyzed to release silanols in a physiological environment.

### Adhesive Latex Formulation

The antimicrobial agent may be used in an adhesive latex formulation. The adhesive latex formulation comprises a polymer, a thickening agent, and the antimicrobial agent.

The antimicrobial agent comprises the silanol containing molecule selected from the group consisting of silanols, siloxanediols and a combination comprising at least one of the foregoing silanol containing molecules.

In one embodiment, the house treating agents may include the antimicrobial agent being released from a silylated polymer. The antimicrobial agent can be released in a controllable manner from the hydrolysis of a silylated polymer. The antimicrobial agent can be in dispersed or bulk form in the silylated polymer. The polymer can be a hydroxyl-containing polymer, where the hydroxyl-containing polymer is a polysaccharide, a protein, a polyalkylene glycol, a hydroxyl-terminated polymer, an amine-terminated polymer, or a combination comprising at least one of the foregoing polymers.

The antimicrobial agent can be released from the deodorant in a controllable manner by hydrolysis of the silylated polymer. The silylation agent can be selected from commercial silylation agents such as Me₃SiNHSiMe₃, Me₃SiCl, Et₃SiNHSiEt₃, PhCH₂CH₂Me₂SiNHSiMe₂CH₂CH₂Ph, other similar precursors to the silanols described above, or a combination comprising at least one of the foregoing silylation agents. The silylated polymer is the reaction product from the hydroxyl containing polymer and the silylation agent; wherein the silylated polymer can be hydrolyzed to release silanols in a physiological environment.

A method of producing the antimicrobial latexes comprises selecting a polymer and compounding it with the antimicrobial agent and a thickener until the resultant composition exhibits a viscosity of at least about 4,000 centipoise at 25° C. and at 1 atmosphere.

As noted above, the compositions containing the antimicrobial agent may be applied to a substrate. Following are several applications where compositions containing the antimicrobial agent are applied to the substrate.

### Medical Graft

In yet another embodiment, the antimicrobial composition may be used in a coating on a porous, flexible, medical graft. The porous, flexible, medical graft comprises (a) fabric material in a generally tubular shape; and (b) an antimicrobial agent silanol comprising a silanol containing molecule selected from the group consisting of silanols, siloxanediols and a combination comprising at least one of the foregoing silanol containing molecules. The silanol containing molecules are bound to the graft via Van de Waals interaction, hydrogen bonding, ionic bonding or covalent bonding. The silanol containing molecules are therefore coated onto the graft and constitutes between 0.01 and 20% of the weight of the graft.

The coating may further comprise an elastomeric material selected from the group consisting of acrylic, polyurethane, silicone, latex, polyglycolic lactic acid, a biodegradable polymer, a hydrophilic biodegradable polymer, and a non-degrading polymer. In one embodiment, the silanol containing molecules are incorporated in 10 nm to 1 mm diameter spheres for prolonged release of the antimicrobial agent at concentrations effective to kill microbes over a period of time. The material for fabricating the spheres can be selected from natural or synthetic, degradable or non-degradable polymers accepted in medical devices.

The antimicrobial agent can be released in a controllable manner from the hydrolysis of a silylated polymer. The antimicrobial agent can be in dispersed or bulk form in the silylated polymer. The polymer can be a hydroxyl-containing polymer, where the hydroxyl-containing polymer is a polysaccharide, a protein, a polyalkylene glycol, a hydroxyl-terminated polymer, an amine-terminated polymer, or a combination comprising at least one of the foregoing polymers.

In one embodiment, the antimicrobial agent can be released in a controllable manner from hydrolysis of the silylated polymer. The silylation agent can be selected from commercial silylation agents such as Me₃SiNHSiMe₃, Me₃SiCl, Et₃SiNHSiEt₃, PhCH₂CH₂Me₂SiNHSiMe₂CH₂CH₂Ph, other similar precursors to the silanols described above, or a combination comprising at least one of the foregoing silylation agents. The silylated polymer is the reaction product from the hydroxyl containing polymer and the silylation agent; wherein the silylated polymer can be hydrolyzed to release silanols in a physiological environment.

The medical graft comprises a fibrous composition and a binder. The fibers of the fabric comprise a polymer selected from the group consisting of organic reinforcing fibrous fillers or synthetic reinforcing fibers. This includes organic polymers capable of forming fibers such as cotton, polyethylene terephthalate, polybutylene terephthalate and other polyesters, polyarylates, polyethylene, polyvinylalcohol, polytetrafluoroethylene, acrylic resins, high tenacity fibers with high thermal stability including aromatic polyamides, polyaramid fibers such as those commercially available from Du Pont de Nemours under the trade name Kevlar, polybenzimidazole, polyimide fibers such as those available from Dow Chemical Co. under the trade names polyimide 2080 and PBZ fiber, polyphenylene sulfide, polyether ether ketone, polyimide, polybenzoxazole, aromatic polyimides or polyetherimides, carbon fibers, and the like. Combinations of any of the foregoing fibers may also be used.

Such reinforcing fillers may be provided in the form of monofilament or multifilament fibers and can be used either alone or in combination with other types of fiber, through, for example, co-weaving or core/sheath, side-by-side, orange-type or matrix and fibril constructions, or by other methods known to one skilled in the art of fiber manufacture. Typical cowoven structures include glass fiber-carbon fiber, carbon fiber-aromatic polyimide (aramid) fiber, and aromatic polyimide fiber-glass fiber. Fibrous fillers may be supplied in the form of, for example, rovings, woven fibrous reinforcements, such as 0-90 degree fabrics, non-woven fibrous reinforcements such as continuous strand mat, chopped strand mat, tissues, papers and felts and 3-dimensionally woven reinforcements, performs and braids.

In general, the amount of fibrous filler present in the medical graft can be up to about 80 wt %, and specifically from about 20 to about 50 wt %, based on the total weight of the medical graft.

In an exemplary embodiment, glass fibers can be used in the medical graft. Useful glass fibers can be formed from any type of fiberizable glass composition known to those skilled in the art, and include those prepared from fiberizable glass compositions commonly known as “E-glass,” “A-glass,” “C-glass,” “D-glass,” “R-glass,” “S-glass,” as well as E-glass derivatives that are fluorine-free and/or boron-free. Most reinforcement mats comprise glass fibers formed from E-glass and are included in the conductive compositions of this invention.

The medical graft may further comprise a coating of a tissue compatible material on at least an exterior surface of the graft.

The method of manufacturing the medical graft comprises (a) providing porous fabric treated with silanol containing molecules bound to the fabric material, wherein the process results in some or all of the silanol containing molecules being leaching in physiological environment in an antimicrobially effective amount; and (b) forming said fabric piece into a generally tubular shape to form the medical graft.

### Dental Floss

Dental floss generally comprises a filament upon which is disposed a composition that contains the antimicrobial agent. The filament may comprise a polymer that may be selected from the list provided above. The antimicrobial agent is mixed with water and a surfactant and disposed upon the filament. The surfactant may be a polyalkylene glycol.

The antimicrobial agent comprises the silanol containing molecule selected from the group consisting of silanols, siloxanediols and a combination comprising at least one of the foregoing silanol containing molecules.

In manufacturing the dental floss, the antimicrobial agent is dispersed into water and/or polyalkylene oxide by means of any mixing equipment to form a mixture. Heat may be used during the formation of the mixture. The mixture is then applied to the filament by draw-dipping equipment. The floss is then dried. The floss may be treated to be hydrophilic. The floss may be used to deliver the antimicrobial agent to an affected area at the interproximal surfaces of the teeth in the mouth of a human being, whereby said coating dissolves thereby releasing said silanol.

### Use on Garments, Handwear, Footwear or Tape

The antimicrobial agent may also be used on garments, handwear, footwear, tapes (e.g., bandages, tourniquets, and the like), and the like. The antimicrobial agent may be applied as a coating to the garments, handwear, footwear, tapes, and the like. The antimicrobial agent comprises the silanol containing molecule selected from the group consisting of silanols, siloxanediols and a combination comprising at least one of the foregoing silanol containing molecules.

The antimicrobial agent can be released in a controllable manner from the hydrolysis of a silylated polymer. The antimicrobial agent can be in dispersed or bulk form in the silylated polymer. The polymer can be a hydroxyl-containing polymer, where the hydroxyl-containing polymer is a polysaccharide, a protein, a polyalkylene glycol, a hydroxyl-terminated polymer, an amine-terminated polymer, or a combination comprising at least one of the foregoing polymers.

In one embodiment, the antimicrobial agent can be released in a controllable manner from hydrolysis of the silylated polymer. The silylation agent can be selected from commercial silylation agents such as Me₃SiNHSiMe₃, Me₃SiCl, Et₃SiNHSiEt₃, PhCH₂CH₂Me₂SiNHSiMe₂CH₂CH₂Ph, other similar precursors to the silanols described above, or a combination comprising at least one of the foregoing silylation agents. The silylated polymer is the reaction product from the hydroxyl containing polymer and the silylation agent; wherein the silylated polymer can be hydrolyzed to release silanols in a physiological environment.

The antimicrobial agent may be incorporated into an antimicrobial composition that comprises about 0.01 to about 10 wt % of the antimicrobial agent and about 0.1 to about 80 wt % of a suitable thermoplastic polymer. Thermoplastic polymers are listed above. The substrate (garments, handwear, footwear, tapes (e.g., bandages, tourniquets, and the like), is coated with the antimicrobial composition. Following this the substrate with the antimicrobial composition may optionally be dried using heat.

### Antimicrobial Wound Dressing

The antimicrobial agent may also be used in a wound dressing. The antimicrobial agent comprises the silanol containing molecule selected from the group consisting of silanols, siloxanediols, or a combination comprising at least one of the foregoing silanol containing molecules.

In one embodiment, the wound dressing may include the antimicrobial agent being released from a silylated polymer. The antimicrobial agent can be released in a controllable manner from the hydrolysis of a silylated polymer. The antimicrobial agent can be in dispersed or bulk form in the silylated polymer. The polymer can be a hydroxyl-containing polymer, where the hydroxyl-containing polymer is a polysaccharide, a protein, a polyalkylene glycol, a hydroxyl-terminated polymer, an amine-terminated polymer, or a combination comprising at least one of the foregoing polymers.

The antimicrobial agent when used in a wound dressing comprises (a) 0.01% to 5 wt % of the antimicrobial agent along with carrier materials used in wound care.

In one embodiment, the antimicrobial agent can be released in a controllable manner from hydrolysis of the silylated polymer. The silylation agent can be selected from commercial silylation agents such as Me₃SiNHSiMe₃, Me₃SiCl, Et₃SiNHSiEt₃, PhCH₂CH₂Me₂SiNHSiMe₂CH₂CH₂Ph, other similar precursors to the silanols described above, or a combination comprising at least one of the foregoing silylation agents. The silylated polymer is the reaction product from the hydroxyl containing polymer and the silylation agent; wherein the silylated polymer can be hydrolyzed to release silanols in a physiological environment. In one embodiment, the antimicrobial agent can employ a polymer selected from the group consisting of a hydroxyl-containing polymer such as a polysaccharide, a protein, a polyalkylene glycol, a hydroxyl-terminated polymer, an amine-terminated polymer, or a combination comprising at least one of the foregoing polymers.

The wound dressing can be a non-resorbable gauze, a sponge dressing for external use, a hydrophilic wound dressing, an occlusive wound dressing, a hydrogel wound and burn dressing or an interactive wound and burn dressing.

### Absorbent Sanitary Article

The antimicrobial agent may also be used in an absorbent sanitary article. The absorbent sanitary article is capable of absorbing body fluids, which comprises a fibrous matrix including at least one fiber having an outer surface the antimicrobial agent. The antimicrobial agent comprises the silanol containing molecule selected from the group consisting of silanols, siloxanediols, or a combination comprising at least one of the foregoing silanol containing molecules.

In one embodiment, the absorbent sanitary article may include the antimicrobial agent being released from a silylated polymer. The antimicrobial agent can be released in a controllable manner from the hydrolysis of a silylated polymer. The antimicrobial agent can be in dispersed or bulk form in the silylated polymer. The polymer can be a hydroxyl-containing polymer, where the hydroxyl-containing polymer is a polysaccharide, a protein, a polyalkylene glycol, a hydroxyl-terminated polymer, an amine-terminated polymer, or a combination comprising at least one of the foregoing polymers.

In one embodiment, the antimicrobial agent can be released in a controllable manner from hydrolysis of the silylated polymer. The silylation agent can be selected from commercial silylation agents such as Me₃SiNHSiMe₃, Me₃SiCl, Et₃SiNHSiEt₃, PhCH₂CH₂Me₂SiNHSiMe₂CH₂CH₂Ph, other similar precursors to the silanols described above, or a combination comprising at least one of the foregoing silylation agents. The silylated polymer is the reaction product from the hydroxyl containing polymer and the silylation agent; wherein the silylated polymer can be hydrolyzed to release silanols in a physiological environment.

In one embodiment, the antimicrobial agent can employ a polymer selected from the group consisting of a hydroxyl-containing polymer such as a polysaccharide, a protein, a polyalkylene glycol, a hydroxyl-terminated polymer, an amine-terminated polymer, or a combination comprising at least one of the foregoing polymers.

In one embodiment, the antimicrobial agent can be used in an amount of 0.001% to 10% of the article on a dry basis. In another embodiment, the antimicrobial agent is used on a fiber, where the fiber is a natural or synthetic fiber accepted by the industry. The fiber may be a discrete fiber, a woven, a non-woven or a thread.

### Lens Care

The antimicrobial agent may also be used in a lens care device. A lens structured to be used in the mammalian eye comprises a lens body including any ophthalmologically accepted polymeric material and the antimicrobial agent.

In one embodiment, the lens care device may include the antimicrobial agent being released from a silylated polymer. The antimicrobial agent can be released in a controllable manner from the hydrolysis of a silylated polymer. The antimicrobial agent can be in dispersed or bulk form in the silylated polymer. The polymer can be a hydroxyl-containing polymer, where the hydroxyl-containing polymer is a polysaccharide, a protein, a polyalkylene glycol, a hydroxyl-terminated polymer, an amine-terminated polymer, or a combination comprising at least one of the foregoing polymers.

In another embodiment, the antimicrobial agent can be released in a controllable manner from hydrolysis of the silylated polymer. The silylation agent can be selected from commercial silylation agents such as Me₃SiNHSiMe₃, Me₃SiCl, Et₃SiNHSiEt₃, PhCH₂CH₂Me₂SiNHSiMe₂CH₂CH₂Ph, other similar precursors to the silanols described above, or a combination comprising at least one of the foregoing silylation agents. The silylated polymer is the reaction product from the hydroxyl containing polymer and the silylation agent; wherein the silylated polymer can be hydrolyzed to release silanols in a physiological environment.

In yet another embodiment, the antimicrobial agent can employ a polymer selected from the group consisting of a hydroxyl-containing polymer such as a polysaccharide, a protein, a polyalkylene glycol, a hydroxyl-terminated polymer, an amine-terminated polymer, or a combination comprising at least one of the foregoing polymers.

The antimicrobial agent is blended with an organic polymeric material to form a lens body. The antimicrobial lens contains at least about 0.005 wt % of the antimicrobial agent and less than about 10 wt % of the antimicrobial agent. An antimicrobial aqueous solution for the care of the antimicrobial contact lens comprises about 0.01% to about 10 wt % of the antimicrobial composition, based upon the weight of the antimicrobial aqueous solution and water or artificial tear.

The blending of the aforementioned ingredients to form the respective compositions involves the use of shear force, extensional force, compressive force, ultrasonic energy, electromagnetic energy, thermal energy or combinations comprising at least one of the foregoing forces or forms of energy, and is conducted in processing equipment wherein the aforementioned forces or forms of energy are exerted by a single screw, multiple screws, intermeshing co-rotating or counter rotating screws, non-intermeshing co-rotating or counter rotating screws, reciprocating screws, screws with pins, screws with screens, barrels with pins, rolls, rams, helical rotors, or combinations comprising at least one of the foregoing.

Blending involving the aforementioned forces may be conducted in machines such as single or multiple screw extruders, Buss kneaders, Henschel mixers, helicones, Ross mixers, Banbury, roll mills, molding machines such as injection molding machines, vacuum forming machines, blow molding machines, or the like, or a combination comprising at least one of the foregoing machines.

In one embodiment, the compositions may be prepared by pre-combining (dry-blending) the antimicrobial agent prior to being fed into a melt blending device, although such pre-combining may not always be desired. The pre-combining may be carried out in a mixer such as, for example, a drum mixer, ribbon mixer, vertical spiral mixer, Muller mixer, sigma mixer, chaotic mixer, static mixer, and the like. Pre-combining is generally carried out at room temperature.

A melt blend is one where at least a portion of a thermoplastic polymer used in the respective compositions has reached a temperature greater than or equal to about its melting temperature, if the thermoplastic polymer is a semi-crystalline organic polymer, or its flow point (e.g., the glass transition temperature) if the thermoplastic polymer is an amorphous organic polymer during the blending process. A dry blend is one where the entire mass of the thermoplastic polymer is at a temperature less than or equal to about its melting temperature, or at a temperature less than or equal to its flow point if the thermoplastic polymer is an amorphous polymer and wherein the thermoplastic polymer is substantially free of any liquid-like fluid during the blending process. Solution blending may also be performed if desired.

In one embodiment, the antimicrobial agent along with other ingredients are added to an extruder and undergo melt blending in the extruder. The extrudate is collected and subjected to molding. The aforementioned ingredients may be added to the throat of the extruder or some of them may be added downstream.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention.

What is claimed is:

1. An oral care composition, comprising:
about 10% to about 99% of at least one ingredient selected from the group consisting of a polishing agent (abrasive agent), sudsing agents (surfactants), a binder, a humectant, a medicinal agent, peroxide sources, alkali metal bicarbonate salts, thickening materials, water, titanium dioxide, flavor agents, sweetening agents, xylitol, coloring agents, water and mixtures thereof; and
an effective amount of an antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6.

2. The oral care composition of claim 1, where the silanol containing molecule is released in a controllable manner by the hydrolysis of a silylated polymer.

3. The oral care composition of claim 2, where the silylated polymer comprises a silylation agent; the silylation agent being Me₃SiNHSiMe₃, Me₃SiCl, Et₃SiNHSiEt₃, or PhCH₂CH₂Me₂SiNHSiMe₂CH₂CH₂Ph.

4. The oral care composition of claim 3, where the silylated polymer comprises hydroxyl groups.

5. A method of manufacturing a oral care composition comprising:
blending together about 10% to about 99% of at least one ingredient selected from the group consisting of a polishing agent, a surfactant, a binder, a humectant, a medicinal agent, a peroxide source, an alkali metal bicarbonate salt, a thickening agent, water, titanium dioxide, a flavor agent, a sweetening agent, xylitol, a coloring agent, and mixtures thereof; and
an effective amount of an antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6.

6. A personal care composition, comprising:
an effective amount of an antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6;
a safe and effective amount of a second active agent, wherein said second active agent is selected from the group consisting of erythritol, p-cymen-7-ol, benzyl phenylacetate, 4-(4-methoxyphenyl)butan-2-one, ethoxyquin, tannic acid, gallic acid, octadecenedioic acid, p-cymen-5-ol, hesperedin, mustard seed extract, glycyrrhizic acid, glycyrrhetinic acid, carnosine, butylated hydroxytoluene, butylated hydroxyanisole, tetrahydrocurcumin, cetyl pyridinium chloride, ergothioneine, vanillin or its derivatives, diethylhexyl syrinylidene malonate, melanostatine, sterol esters, creatine, creatinine, feverfew extract, licochalcone A, sugar amine, vitamin B3 compounds, retinoids, peptides, phytosterol, dialkanoyl hydroxyproline, hexamidine compounds, salicylic acid, n-acyl amino acid compounds, sunscreen actives, water soluble vitamins, oil soluble vitamins, yeast cell derivative, and combinations thereof;
an additional component, wherein the additional component is selected from the group consisting of desquamatory actives, anti-acne actives, wrinkle repair actives, anti-oxidants, radical scavengers, chelators, flavonoids, anti-inflammatory agents, anti-cellulite agents, tanning actives, skin lightening agents, antimicrobial actives, antifungal actives, conditioning agents, thickening agents, particulate material, topical anesthetics, and combinations thereof;
and a dermatologically acceptable carrier.

7. The personal care composition of claim 6, where the silanol containing molecule is released in a controllable manner by the hydrolysis of a silylated polymer.

8. The personal care composition of claim 7, where the silylated polymer comprises a silylation agent; the silylation agent being Me₃SiNHSiMe₃, Me₃SiCl, Et₃SiNHSiEt₃, or PhCH₂CH₂Me₂SiNHSiMe₂CH₂CH₂Ph.

9. The personal care composition of claim 8, where the silylated polymer comprises hydroxyl groups.

10. An adhesive latex formulation comprising:
a polymer;
a thickening agent; and
an effective amount of an antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6.

11. The adhesive latex formulation composition of claim 10, where the silanol containing molecule is released in a controllable manner by the hydrolysis of a silylated polymer.

12. The adhesive latex formulation composition of claim 11, where the silylated polymer comprises a silylation agent; the silylation agent being Me₃SiNHSiMe₃, Me₃SiCl, Et₃SiNHSiEt₃, or PhCH₂CH₂Me₂SiNHSiMe₂CH₂CH₂Ph.

13. The adhesive latex formulation composition of claim 12, where the silylated polymer comprises hydroxyl groups.

14. The adhesive latex formulation composition of claim 10, comprising about 0.01 and to about 20 wt % of the antimicrobial agent based on the total weight of the adhesive latex formulation.

15. The adhesive latex formulation composition of claim 14, where the latex formulation is used in a household treating agent selected from the group consisting of decorative preparations, laundry detergents, fabric softeners, non-detergent based fabric care products, liquid cleansing and scouring agents, glass detergents, neutral cleaners, all-purpose cleaners, acid household cleaners, bathroom cleaners, dishwashing agents, kitchen and oven cleaners, clear rinsing agents, dishwasher detergents, shoe polishes, polishing waxes, floor detergents and polishes, metal, glass, and ceramic cleaners, fabric care products, rug cleaners and carpet shampoos, agents for removing rust, furniture and multipurpose polishes, leather and vinyl dressing agents, and solid and liquid air fresheners.

16. A method comprising:
blending together a polymer; a thickening agent; and an effective amount of an antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6 to form a adhesive latex formulation composition; the adhesive latex formulation having a viscosity of at least 4,000 centipoise at 25° C. and at 1 atmosphere.

17. The method of claim 16, where blending is conducted in a device that employs shear force, extensional force, compressive force, ultrasonic energy, electromagnetic energy, thermal energy or combinations comprising at least one of the foregoing forces or forms of energy.

18. The method of claim 17, where the blending is conducted in devices that contain a single screw, multiple screws, intermeshing co-rotating or counter rotating screws, non-intermeshing co-rotating or counter rotating screws, reciprocating screws, screws with pins, screws with screens, barrels with pins, rolls, rams, helical rotors, or combinations comprising at least one of the foregoing.

19. A household treating agent comprising:
about 0.01 wt % to 20 wt % of an antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6.

20. The household treating agent of claim 19, where the silanol containing molecule is released in a controllable manner by the hydrolysis of a silylated polymer.

21. The household treating agent composition of claim 20, where the silylated polymer comprises a silylation agent; the silylation agent being Me₃SiNHSiMe₃, Me₃SiCl, Et₃SiNHSiEt₃, or PhCH₂CH₂Me₂SiNHSiMe₂CH₂CH₂Ph.

22. The household treating agent composition of claim 21, where the silylated polymer comprises hydroxyl groups.

23. A deodorant composition comprising:
a deodorant;
an antimicrobial agent;
a polymer; and
an aqueous or oily liquid medium; wherein the antimicrobial agent comprises a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6.

24. The deodorant composition of claim 23, further comprising a dermatologically accepted fragrance.

25. The deodorant composition of claim 23, further comprising a dermatologically acceptable carrier.

26. A dental floss article comprising:
a filament;
an antimicrobial agent; the antimicrobial agent being disposed upon the filament; the antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6.

27. A dental floss composition comprising:
water;
a surfactant; and
an antimicrobial agent; the antimicrobial agent being disposed upon the filament; the antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6.

28. A method comprising:
blending together water; a surfactant; and an antimicrobial agent to form a dental composition; the antimicrobial agent being disposed upon the filament; the antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6;
applying the dental composition to the filament; wherein applying the dental composition to the filament is accomplished in a draw-dipping equipment; and
drying the dental composition.

29. A medical graft comprising:
a fabric material in a tubular shape; and
an antimicrobial agent the antimicrobial agent being disposed upon the filament; the antimicrobial agent comprising a silanol containing molecule, the silanol containing molecule comprising silanols (R₁R₂R₃SiOH), siloxanediols HO(R₁R₂SiO)ₙH, siloxanols HO(R₁R₂SiO)ₙSiR₁R₂R₃ or combinations thereof, where R₁, R₂ and R₃ are selected from alkyl or fluoroalkyl moieties having 1 to 4 carbon atoms, vinyl or aryl groups and n is less than about 6.

30. The medical graft of claim 29, where the fabric material comprises a fibrous composition and a binder.

31. The medical graft of claim 30, where the fibrous composition comprises organic reinforcing fibrous fillers or synthetic reinforcing fibers.

32. The medical graft of claim 30, where the fibrous composition comprises cotton, polyethylene terephthalate, polybutylene terephthalate, polyesters, polyarylates, polyethylene, polyvinylalcohol, polytetrafluoroethylene, acrylic resins, aromatic polyamides, polyaramid fibers, polybenzimidazole, polyphenylene sulfide, polyether ether ketone, polyimide, polybenzoxazole, aromatic polyimides or polyetherimides, glass fibers, carbon fibers, or combinations thereof.

33. The medical graft of claim 30, wherein the antimicrobial agent is disposed on the fabric material in the form of a coating.

34. The medical graft of claim 30, where the silanol containing molecule is released in a controllable manner by the hydrolysis of a silylated polymer.

35. The adhesive latex formulation composition of claim 34, where the silylated polymer comprises a silylation agent; the silylation agent being Me₃SiNHSiMe₃, Me₃SiCl, Et₃SiNHSiEt₃, or PhCH₂CH₂Me₂SiNHSiMe₂CH₂CH₂Ph.

36. The adhesive latex formulation composition of claim 35, where the silylated polymer comprises hydroxyl groups.

37. A lens care device comprising the antimicrobial agent.

38. An absorbent sanitary article comprising the antimicrobial agent.

39. An antimicrobial wound dressing comprising the antimicrobial agent.
